(19) United States
(12) Patent Application Publication
(10) Pub. No.: US 2020/0121936 A1
(43) Pub. Date: Apr. 23, 2020

(54) COIL ARRANGEMENT FOR A PROGRAMMING DEVICE AND PROGRAMMING DEVICE

(71) Applicant: BIOTRONIK SE & CO. KG, Berlin (DE)

(72) Inventor: Julian Merlin, Berlin (DE)

(21) Appl. No.: 16/593,556

(22) Filed: Oct. 4, 2019

(30) Foreign Application Priority Data
Oct. 17, 2018 (EP) 18200967.0

(51) Int. Cl. A61N 1/372; H01F 38/14; H01F 27/34; A61N 1/39; H04B 5/00

(52) U.S. Cl. CPC A61N 1/37229; H01F 38/14; H01F 2038/143; A61N 1/3956; H04B 5/0081; H01F 27/34

(57) ABSTRACT

A coil arrangement for a programming device has a transmitting coil that is set up to emit a transmitted signal, and multiple receiving coils that are set up to receive a received signal. The transmitting coil and the receiving coils are arranged such that the magnetic fluxes generated in the receiving coils by a transmitted signal emitted from the transmitting coil essentially cancel out each other or the voltages induced in the receiving coils by a transmitted signal emitted from the transmitting coil essentially cancel out each other. In addition, a programming device has such a coil arrangement.

### CROSS-REFERENCE TO RELATED APPLICATION

This application claims the priority, under 35 U.S.C. § 119, of European application EP 18200967.0, filed Oct. 17, 2018; the prior application is herewith incorporated by reference in its entirety.

### BACKGROUND OF THE INVENTION

### Field of the Invention

The disclosure relates to a coil arrangement for a programming device and a programming device.

Some medical implants (e.g., cardiac pacemakers or cardioverter-defibrillators) can communicate with a programming device, for example in order send measurements from the implant to the programming device and/or to transfer device parameters from the programming device to the implant. Usually, the communication occurs by use of electromagnetic fields.

In the case of communications systems that work in both directions, transmitters and receivers must work either on the same antennas or on different antennas that lie close together. Generally, this requires switch-over mechanisms or other precautions to prevent the transmit power entering the input of the receiver and being consumed there, instead of being emitted. When magnetic loop antennas lie close together, strong fields are coupled into the receiver during transmission if there is no compensation. To protect the receiver, protective diodes must be used or other measures must be taken to limit voltage or current, if there should not be any active switchover. Furthermore, it is often necessary to place an electrical load on the receiving coils, i.e.; to terminate them with a sufficiently low resistance to limit increases in voltage at the self-resonant frequency. Usually, these measures lead to a not insignificant loss of energy from the transmission field, thereby reducing the range. Resonance in the receiving coils can also cause feedback on the transmission field. Generally, it is also impossible to transmit and receive simultaneously without compensation of the voltages induced in the receiving coils during transmission, since then the receiver will be extremely overdriven.

U.S. Pat. No. 5,630,835 A describes some methods to decouple the transmitter and the receiver. These involve coils being switched over or turned off. In one embodiment, an antenna has two coils which are arranged in such a way that the effects of far-field interference signals on received near field signals are suppressed.

German patent DE 199 28 216 C2, corresponding to U.S. Pat. No. 6,405,088, discloses a telemetry coil arrangement to receive data signals, especially data signals from cardiac implants. The arrangement comprises a multi-coil bobbin with four coils that are coaxially arranged one after the other. One of the four coils is a transmitting coil. The other coils are variably connectable into a receiving coil system.

U.S. Pat. No. 5,741,315 A discloses a device for receiving telemetry signals from an implant. A coil receives both usable signal components and parasitic signal components. A compensating coil is used to suppress the parasitic signal components.

U.S. Pat. No. 8,847,434 B2 discloses an antenna arrangement with a compensating coil to reduce a transmitted signal in a receiver.

### SUMMARY OF THE INVENTION

The objective is to provide improved technologies for communication between two devices, especially between a programming device and an implant. An effect of a transmitted signal on a receiving circuit should be reduced.

A coil arrangement for a programming device and a programming device according to the independent claims are disclosed. Further embodiments are subject matter of the dependent claims.

According to one aspect, a coil arrangement for a programming device is provided. The coil arrangement has a transmitting coil configured to emit a transmitted signal, and multiple receiving coils configured to receive a received signal. The transmitting coil and the receiving coils are arranged such that the magnetic fluxes produced in the receiving coils by a transmitted signal emitted from the transmitting coil essentially cancel out each other or the voltages induced in the receiving coils by a transmitted signal emitted from the transmitting coil essentially cancel out each other.

According to a further aspect, a programming device for an implant is provided with a coil arrangement disclosed here.

It is also possible to provide a system with a programming device and a medical implant. The medical implant may be an implantable cardiac pacemaker, an implantable cardioverter-defibrillator (ICD), or an implantable sensor. The programming device may be configured to communicate with the implant. For example, measurements may be transferred from the implant to the programming device and/or device parameters may be sent from the programming device to the implant.

The receiving coils may form a receiving coil system that comprises two, three, or even more receiving coils. The receiving coils may be coupled (connected) together. When the transmitting coil emits the transmitted signal, it produces an electromagnetic field that flows through the receiving coils of the receiving coil system. The transmitting coil is arranged such that the magnetic flux through the receiving coils of the receiving system connected in the opposite direction is of the same magnitude, and so in sum becomes zero, or the receiving coils have different diameters and numbers of turns, so the induced voltages are cancelled out. Mostly, the cancelling is not perfectly zero due to mechanical tolerances during assembly of the coils and windings. For the magnetic flux applies that the total amount of the magnetic flux through the oppositely interconnected receiving coils is at least 100 times less than the amount of the magnetic flux in the first receiving coil. The voltage at the receiver, i.e., at the output of the receiving coil system, is (e.g., in the embodiment according to FIG. 3) at least 100 times lower than the voltage induced in a single receiving coil at the same distance from the transmitting coil.

By the inventive coil arrangement, a loss of transmit power due to coupling in receiving coils is avoided. Furthermore, strong overdriving of the receiver is avoided.

The receiving coil system may be configured according to the needs of reception, for example, to suppress far-field sources of interference and/or to maximize the received power in a certain direction (directional characteristics).

The receiving coils may comprise a first receiving coil and a second receiving coil. The first receiving coil and a second receiving coil may be connected together. In one embodiment, exactly two receiving coils may be provided. The transmitted signal of the transmitting coil induces a first voltage in the first receiving coil and a second voltage in the second receiving coil. The transmitting coil, the first receiving coil, and the second receiving coil may be arranged such that a difference between the first voltage and the second voltage is almost zero. If the first receiving coil and the second receiving coil have the same shape and the same number of turns, a first magnetic flux generated with the transmitted signal in the first receiving coil and a second magnetic flux generated with the transmitted signal in the second receiving coil have essentially the same magnitude and almost cancel out each other.

The first receiving coil may be arranged on a first side of the transmitting coil and the second receiving coil may be arranged on a second side of the transmitting coil, the first side lying opposite the second side. In other words, the transmitting coil, the first receiving coil, and the second receiving coil may be arranged in three planes that are different from one another. The three planes may be parallel to one another. For example, the transmitting coil may be centrally arranged between the two receiving coils, the first receiving coil being arranged above the transmitting coil and the second receiving coil being arranged beneath the transmitting coil.

It may be provided that the distance between the transmitting coil and the first receiving coil and the distance between the transmitting coil and the second receiving coil are the same. Alternatively, the distance between the transmitting coil and the first receiving coil and the distance between the transmitting coil and the second receiving coil may be different.

According to another embodiment, it may be provided that the transmitting coil lies in a first plane and the first receiving coil and the second receiving coil lie in a second common plane, the first plane being different from the second plane. The first plane and the second plane may be parallel to one another.

It may also be provided that the transmitting coil, the first receiving coil, and the second receiving coil lie in a common plane.

The first receiving coil and the second receiving coil may be coaxially arranged. It may be provided that the transmitting coil, the first receiving coil, and the second receiving coil are coaxially arranged.

If all coils (the transmitting coil, the first receiving coil, and the second receiving coil) lie coaxially in one plane, a good transmit signal suppression is possible with good far-field suppression, if the transmitting coil is substantially larger than the two receiving coils. The field inside the transmitting coil is quite homogeneous, but not perfectly homogeneous as interfering far fields are. Therefore, with this arrangement it is difficult to achieve optimal suppression of the transmission field and the far field simultaneously. The larger the transmitting coil is in comparison with the two receiving coils, the better the simultaneous suppression that can be achieved.

The first receiving coil and the second receiving coil may be identical, that is, in particular, they may have the same number of turns, the same shape (e.g., have a circular or a rectangular cross section), the same dimensions, and the same materials. This embodiment may be designated as a symmetrical double coil system. The first receiving coil and the second receiving coil may also be different. In this case, the properties (number of turns, shape, dimension and/or material) of the two receiving coils should be matched with one another such that during transmission the voltages induced in the receiving coils (essentially) cancel out each other. The numbers of turns of the two receiving coils are configured such that in the case of transmission the flux in the two receiving coils multiplied by the number of turns of the respective coils is of the same magnitude. Other criteria for the receiving coils result from the desired directional characteristics and position of directional nulls. Further embodiments relating to the parameters of the receiving coils are described in German patent DE 199 28 216 C2.

Other features which are considered as characteristic for the invention are set forth in the appended claims.

Although the invention is illustrated and described herein as embodied in a coil arrangement for a programming device and a programming device, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE SEVERAL VIEWS OF THE DRAWING

FIG. 1 is a diagrammatic, perspective view of a programming head of a programming device according to the invention;

FIG. 2 is a side view of the programming head from FIG. 1;

FIG. 3 is a side view (upper picture of FIG. 3), and a top view (lower picture of FIG. 3) of a coil arrangement;

FIG. 4 is an illustration showing a field line pattern for the coil arrangement according to FIG. 3, when signals are received from an implant;

FIG. 5 is an illustration showing the field line pattern for the coil arrangement according to FIG. 3, when signals are transmitted to an implant;

FIG. 6 is a side view of another embodiment of a coil arrangement;

FIG. 7 is an illustration showing the field line pattern for the coil arrangement according to FIG. 6, when signals are transmitted to an implant;

FIG. 8 is a side view of yet another embodiment of a coil arrangement;

FIG. 9 is a side view of yet another embodiment of a coil arrangement;

FIG. 10 is a side view (upper picture of FIG. 10) and a top view (lower picture of FIG. 10) of another embodiment of a coil arrangement; and

FIG. 11 is a circuit diagram with two receiving coils and one transmitting coil.

### DETAILED DESCRIPTION OF THE INVENTION

In the following discussion, the same reference numbers are used for the same components.

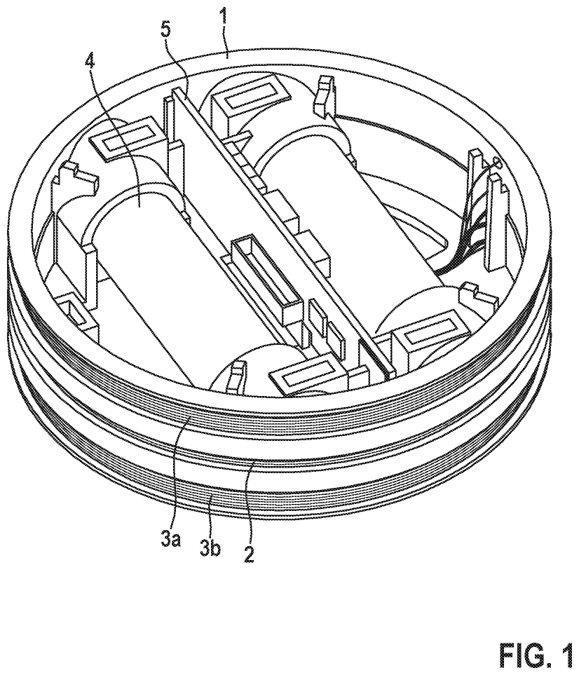

Referring now to the figures of the drawings in detail and first, particularly to FIG. 1 thereof, there is shown a programming head of a programming device of the invention with a coil arrangement. A bobbin (coil former) 1 has a transmitting coil 2, a first receiving coil 3a, and a second receiving coil 3b wound on it. Each of the coils 2, 3a, 3b forms a circle. The bobbin 1 has a magnet 4 (e.g., a permanent magnet) and a printed circuit board 5 arranged inside it. The printed circuit board 5 has electronic components (e.g., a processor and memory) for operating the programming device arranged on it.

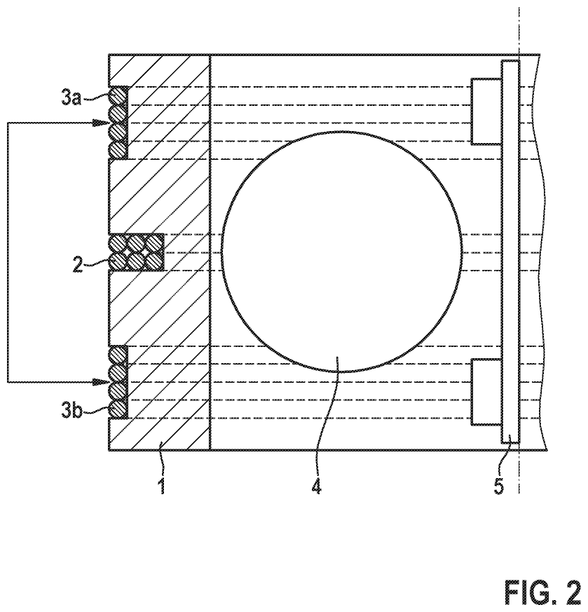

The transmitting coil 2 is centrally arranged between the first receiving coil 3a and the second receiving coil 3b (FIG. 2). The distance between the transmitting coil 2 and the first receiving coil 3a is the same as the distance between the transmitting coil 2 and the second receiving coil 3b. For the transmitting coil 2, the number of turns is n=The first receiving coil 3a and the second receiving coil 3b have an identical structure. Both receiving coils 3a, 3b have the same diameter and the same number of turns (e.g., n=40).

The transmitting coil 2, the first receiving coil 3a, and the second receiving coil 3b are arranged such that when a signal is transmitted from the transmitting coil 2 to the two receiving coils 3a, 3b, two fields of equal magnitude and opposite direction are generated. Thus, the sum of the field in the first receiving coil 3a and the field in the second receiving coil 3b is almost zero. Deviations from the exact value of zero may result on the basis of manufacturing tolerances of the components.

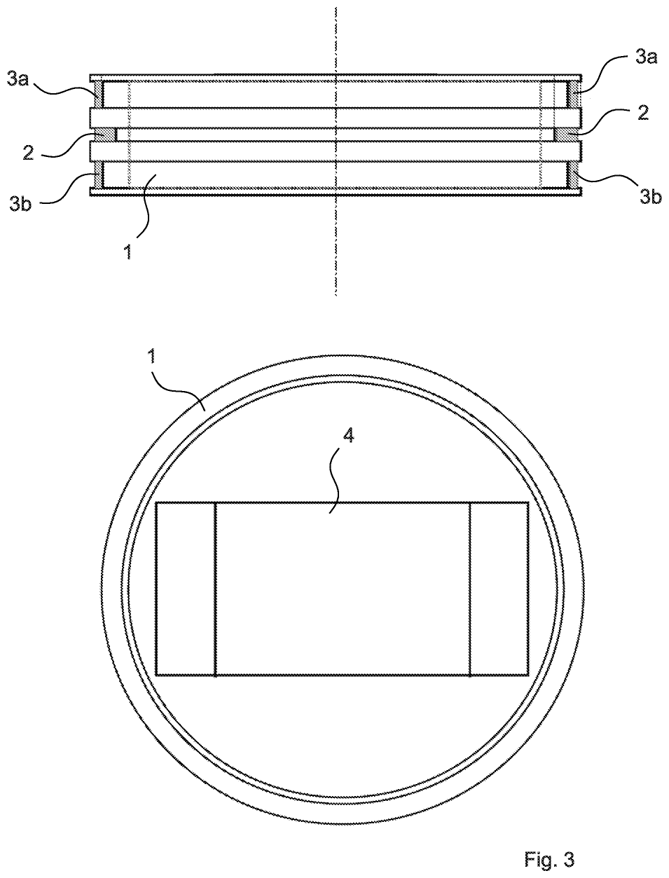

FIG. 3 shows an embodiment similar to that in FIG. 1. The transmitting coil 2 (e.g., with a number of turns n=14) is centrally arranged between two identical receiving coils 3a, 3b. Each receiving coil 3a, 3b can have a number of turns n=60, each comprising two layers of 30 turns. The magnet 4 is arranged in the middle of the coil former 1.

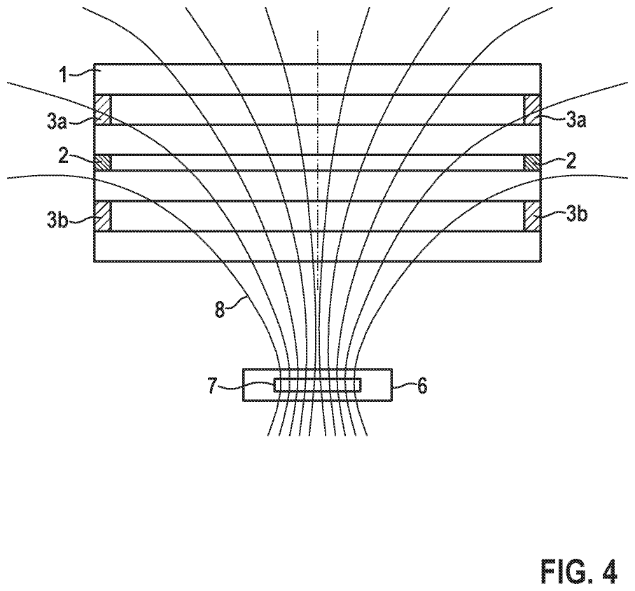

FIG. 4 is a schematic illustration of exemplary field lines courses 8 for the case, in which an implant 6 (with an implant coil 7) transmits a signal to the programming device. This is the receiving case for the coil arrangement. The fluxes in the first receiving coil 3a are different from those in the second receiving coil 3b. Thus, the voltage induced in the two receiving coils 3a, 3b is also different. The difference of the voltages induced in the two receiving coils 3a, 3b is fed to a receiver (not shown). This is the so-called double coil principle.

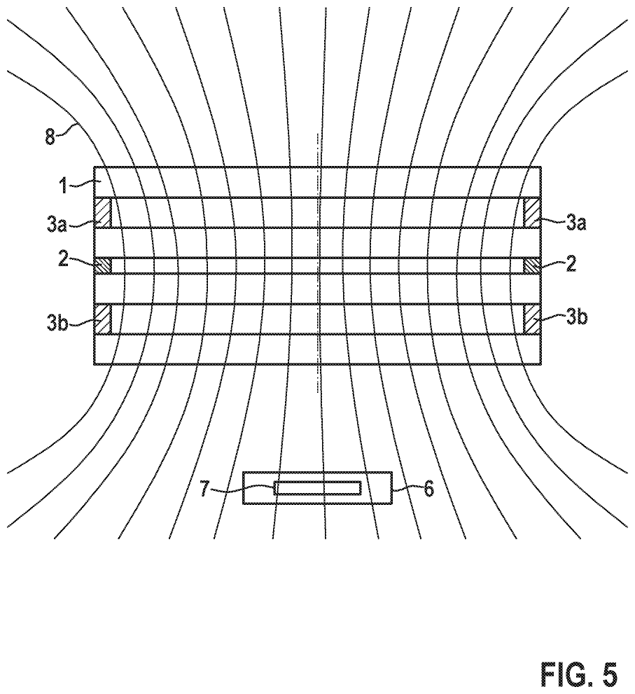

FIG. 5 is a schematic illustration of the field lines 8 for the case in which the transmitting coil 2 of the programming device is used to transmit a signal to the implant 6 (transmission case). Due to the central position of the transmitting coil 2 between the two receiving coils 3a, 3b the flux and the voltage in these two receiving coils 3a, 3b are of the same magnitude and opposite direction. The differential voltage fed to the receiver is zero.

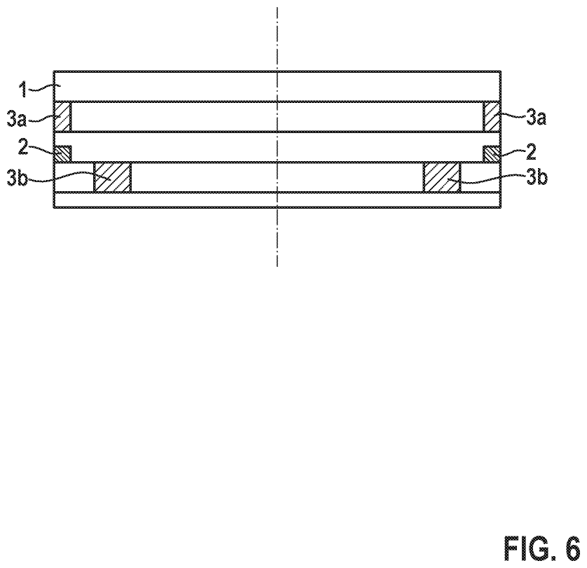

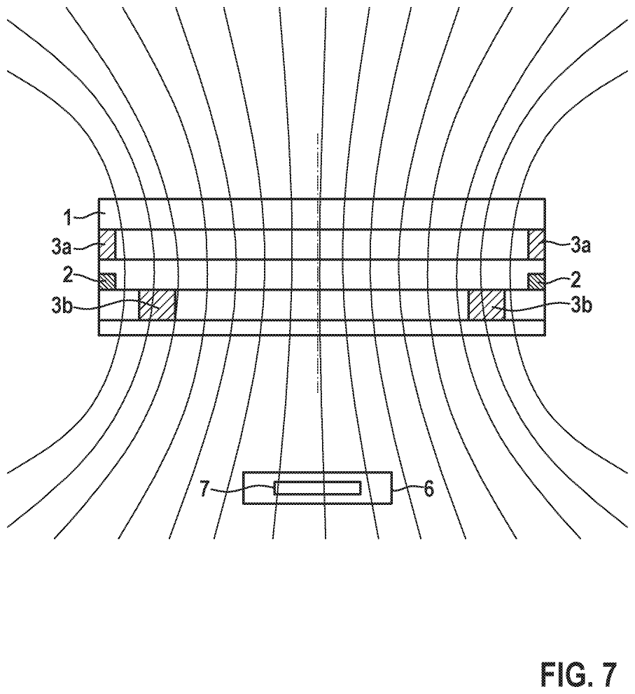

FIG. 6 shows an embodiment with asymmetric receiving coils. The first receiving coil 3a has a larger diameter than the second receiving coil 3b. The diameters and numbers of turns of the two receiving coils 3a, 3b are configured in such a way that in the case of transmission the flux in the two receiving coils 3a, 3b multiplied by the number of turns of the respective coils is of the same magnitude. This makes the voltage difference fed to the receiver equal to zero. Sample field lines 8 during transmission to an implant 6 are shown in FIG. 7.

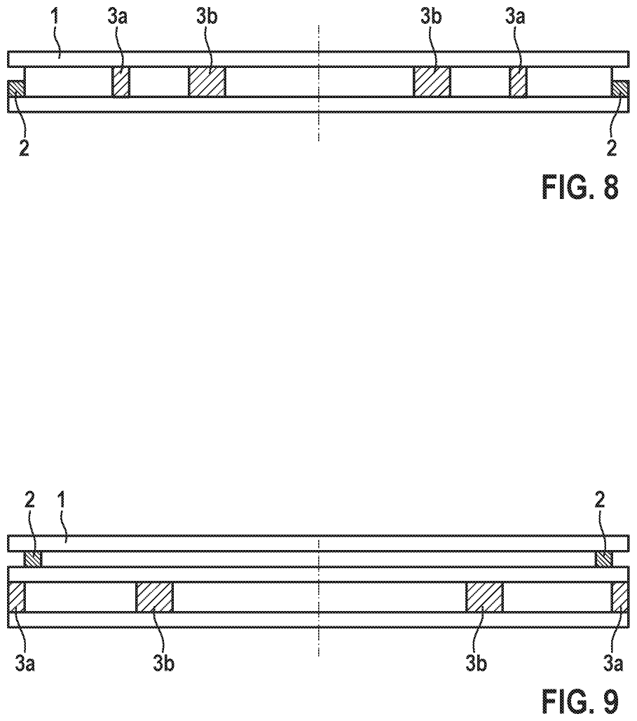

FIG. 8 shows an embodiment in which all three coils (transmitting coil 2, first receiving coil 3a, and second receiving coil 3b) are arranged in a common plane. The first receiving coil 3a has a larger diameter than the second receiving coil 3b. The diameter of the transmitting coil 2 is greater than the diameter of the first receiving coil 3a. The second receiving coil 3b has a larger number of turns than the first receiving coil 3a, The diameters and numbers of turns of the two receiving coils 3a, 3b are configured such that in the case of transmission the flux in the two receiving coils 3a, 3b multiplied by the number of turns of the respective coils is of the same magnitude, or that the flux of a homogeneous far field in the two receiving coils 3a, 3b multiplied by the respective number of turns is of the same magnitude. In this arrangement, the design of the numbers of turns can achieve either an optimal far-field suppression or a minimal transmission field coupling, or a compromise.

In the embodiment shown in FIG. 9, the two receiving coils 3a, 3b are arranged in a common first plane and the transmitting coil 2 is arranged in a second plane, the first plane being parallel to the second plane. The first receiving coil 3a has a larger diameter than the second receiving coil 3b.

In the embodiment shown in FIG. 1 through 9, the transmitting coil 2, the first receiving coil 3a, and the second receiving coil 3b are each circular and coaxially arranged.

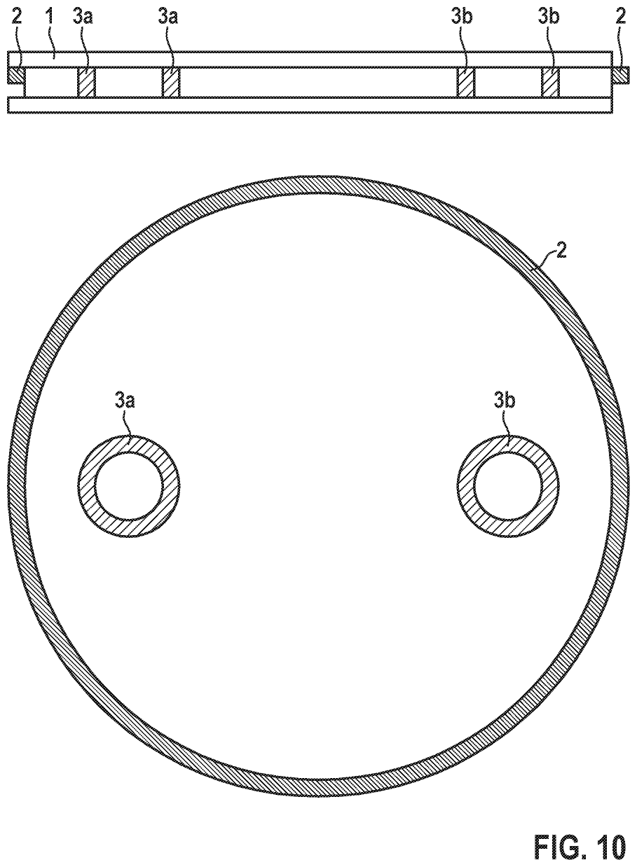

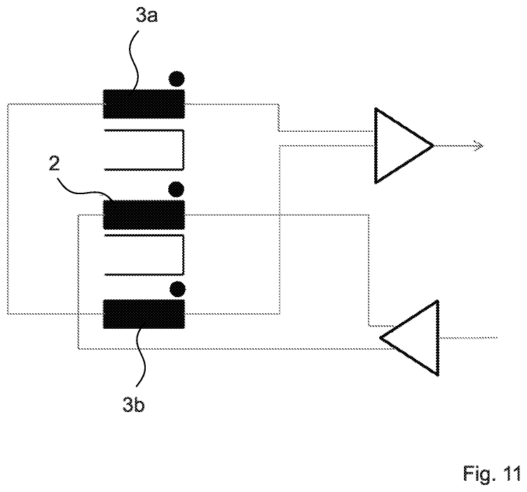

FIG. 10 shows another embodiment. The transmitting coil 2, the first receiving coil 3a, and the second receiving coil 3b are arranged in a common plane. The two receiving coils 3a, 3b are circular, however they are not coaxially arranged. Both receiving coils 3a, 3b have the same diameter and are arranged adjacent to each other inside the transmitting coil 2.

In all disclosed embodiments, the first receiving coil 3a and the second receiving coil 3b are coupled together. FIG. 11 shows a schematically depicted wiring or circuitry of the transmitting coil 2, the first receiving coil 3a, and the second receiving coil 3b as it may be used in all disclosed embodiments.

With the embodiments disclosed here it is possible to achieve the following advantages:

a) compensation of the transmission fields at the receiver without additional compensating coils or circuits;
b) allowing or improving the full-duplex operation (simultaneous transmission and reception); and/or
c) receiving coils may have electrical loads placed on them without distorting or weakening the transmission field, and do not need to be released in the case of transmission.

The features disclosed in the description, the claims, and the figures may be relevant, both individually and in any combination with one another, for realization of the embodiments.

The following is a summary list of reference numerals and the corresponding structure used in the above description of the invention:
1 Bobbin (coil former)
2 Transmitting coil
3a First receiving coil
3b Second receiving coil
4 Magnet
5 Printed circuit board
6 Implant
7 Implant coil
8 Field lines

1. A coil configuration for a programming device, the coil configuration comprising:
a transmitting coil configured to emit a transmitted signal;
a plurality of receiving coils configured to receive a received signal;
said transmitting coil and said receiving coils being disposed such that:
magnetic fluxes generated in said receiving coils by a transmitted signal emitted from said transmitting coil essentially cancel out each other; or
voltages induced in said receiving coils by the transmitted signal emitted from said transmitting coil essentially cancel out each other; and
said receiving coils include a first receiving coil and a second receiving coil, wherein:
said transmitting coil, said first receiving coil, and said second receiving coil lie in three planes that are different from one another; or
said transmitting coil lies in a first plane, and said first receiving coil and said second receiving coil lie in a second common plane, the first plane being different from the second plane.

2. The coil configuration according to claim 1, wherein a distance between said transmitting coil and said first receiving coil is a same as a distance between said transmitting coil and said second receiving coil.

3. The coil configuration according to claim 1, wherein said first receiving coil and said second receiving coil are configured identically.

4. The coil configuration according to claim 1, wherein said first receiving coil and said second receiving coil are configured differently.

5. The coil configuration according to claim 1, wherein said first receiving coil and said second receiving coil are coaxially disposed.

6. The coil configuration according to claim 1, wherein said transmitting coil is disposed between said first receiving coil and said second receiving coil.

7. A programming device for an implant, the programming device comprising:
a coil configuration, containing:
a transmitting coil configured to emit a transmitted signal;
a plurality of receiving coils configured to receive a received signal;
said transmitting coil and said receiving coils being arranged such that:
magnetic fluxes generated in said receiving coils by a transmitted signal emitted from said transmitting coil essentially cancel out each other; or
voltages induced in said receiving coils by the transmitted signal emitted from said transmitting coil essentially cancel out each other; and
said receiving coils include a first receiving coil and a second receiving coil, wherein:
said transmitting coil, said first receiving coil, and said second receiving coil lie in three planes that are different from one another; or
said transmitting coil lies in a first plane, and said first receiving coil and said second receiving coil lie in a second common plane, the first plane being different from the second plane.
